(19) United States
(12) Patent Application Publication
(10) Pub. No.: US 2006/0236469 A1
(43) Pub. Date: Oct. 26, 2006

(54) Anhydrous composition in the form of a film comprising a film-forming polymer and oxidizing agent, preparation and process for coloring keratinous fibers employing the anhydrous composition

(76) Inventors: Eric Bone, Tokyo (JP); Jean Cotteret, Verneuil Sur Seine (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/362,790

(22) Filed: Feb. 28, 2006

Related U.S. Application Data
(60) Provisional application No. 60/681,954, filed on May 18, 2005.

(30) Foreign Application Priority Data
Feb. 28, 2005 (FR) 05 02028

(51) Int. Cl. A61K 8/00

(52) U.S. Cl. 8/405

(57) ABSTRACT

The present disclosure relates to an anhydrous composition in the form of a film comprising at least one film-forming polymer and at least one oxidizing agent. The present disclosure also relates to a process for the preparation of such a composition in which a precursor composition comprising, in an appropriate solvent, a mixture comprising at least one oxidizing agent and at least one film-forming polymer is applied to a support and then the solvent is evaporated. The present disclosure also relates to a process for dyeing keratinous fibers which comprises bringing the fibers and the composition into contact in the presence of an aqueous medium.

This application claims benefit of U.S. Provisional Application No. 60/681,954, filed May 18, 2005, the contents of which are incorporated herein by reference. This application also claims benefit of priority under 35 U.S.C. § 119 to French Patent Application No. FR 05 02028, filed Feb. 28, 2005, the contents of which are also incorporated herein by reference.

The present disclosure relates to an anhydrous composition in the form of a film comprising at least one film-forming polymer and at least one oxidizing agent, and also a process for the preparation of the anhydrous composition. The present disclosure also relates to a process for the treatment of keratinous fibers, including human keratinous fibers, employing such a composition, as well as to its use in the dyeing, bleaching or permanent deformation of keratinous fibers.

It is common to modify the appearance of the hair, either its color (e.g., in order to change or mask the white hairs thereof) or alternatively its form.

Two types of known technologies are primarily employed for the dyeing of human keratinous fibers.

The first method, known as direct or semi-permanent dyeing, comprises changing or introducing the color by the application of a colored molecule which penetrates inside the fiber by diffusion and/or which remains adsorbed at its surface. It is also possible to carry out this dyeing in the presence of an alkaline agent under oxidizing conditions. In such a case, lightening of the fibers accompanied by dyeing of the latter are observed simultaneously. The term used is then direct dyeing under lightening conditions.

The second method, known as oxidation dyeing or permanent dyeing, comprises changing or introducing the color by carrying out, within the actual fiber, an oxidative condensation of dye precursors which are colorless or only slightly colored compounds. After this reaction, the dyes formed are insoluble and are trapped inside the fiber.

It is likewise possible to combine these two methods.

The methods summarized above allow access to numerous intense, fairly persistent and not very selective colors.

One of the most common processes for bleaching keratinous fibers comprises applying an oxidizing agent to the fibers which can be chosen from peroxygen compounds or persalts. Depending on the nature of the oxidizing agent, the hair is either lightened or bleached.

With regard to the permanent deformation processes (e.g., curling, smoothing), these treatments are usually carried out in two steps. More specifically, a first stage is carried out in the presence of a reducing agent, during which the disulphide bridges present in the keratinous fibers are opened.

Before, after or simultaneously with the reduction of the disulphide bridges, the fiber is shaped in a desired way (e.g., curling, smoothing).

Once this first stage has been carried out, it is necessary to carry out this stage during which the disulphide bridges are reformed in order to stabilize the form obtained. This stage of fixing the form is carried out using a composition comprising an oxidizing agent.

The present disclosure relates, for example, to the fields of the treatment of keratinous fibers in which the use of an oxidizing composition is necessary.

One of the difficulties encountered in such processes lies in the fact that the oxidizing agents are delicate compounds which are sometimes unstable in aqueous solution.

Thus, their use requires specific packaging, such as bags for powders, for example, or else requires the use of effective stabilizing agents.

Finally, other difficulties may also be encountered due to the fact that the oxidizing compositions are mixed at the time of use, before the application, with other compositions, such as dyeing compositions, or with aqueous compositions comprising additives necessary for the process. Thus, such embodiments result in procedures which are lengthy and sometimes difficult while not guaranteeing perfect homogeneity of the composition to be applied to the fibers.

Thus, there is a desire to solve the abovementioned disadvantages.

A first aspect of the present disclosure thus relates to an anhydrous composition in the form of a film comprising at least one film-forming polymer and at least one oxidizing agent.

The present disclosure also relates to a process for the preparation of such a composition in which a precursor composition comprising, in an appropriate solvent, a mixture comprising at least one oxidizing agent and at least one film-forming polymer is applied to a support and then the solvent is evaporated.

Another aspect of the present disclosure relates to a process for the treatment of keratinous fibers, including human keratinous fibers, comprising bringing the fibers and the anhydrous composition into contact in the presence of an aqueous medium.

The present disclosure also relates to the uses of the anhydrous composition in the dyeing, bleaching or permanent deformation of keratinous fibers.

The anhydrous composition of the present disclosure makes it possible to improve the stability of the oxidizing agents during storage without necessarily having recourse to the use of stabilizing agents. This is because the oxidizing agents are protected by the polymer film.

Furthermore, the conditions for application of the composition can be simplified. This is because the stage of mixing the oxidizing composition with the ancillary composition before the application to the fibers can be dispensed with. It is possible to carry out such a mixing, but the nature of the composition according to the present disclosure is such that the mixing and the homogenization of the compositions may be greatly simplified.

However, other characteristics of the present disclosure will become more clearly apparent on reading the description and examples which will follow.

In the present disclosure, unless otherwise indicated, when a range of values is given, the limits of this range are included in the span thus defined.

The present disclosure relates to the treatment of keratinous fibers, such as, for example, human keratinous fibers, including hair.

Within the meaning of the present disclosure, the composition is regarded as anhydrous when its water content is less than 10% by weight of the composition, such as less than 5% by weight, or less than 3% by weight, relative to the total weight of the composition. In at least one embodiment, the composition does not comprise water.

As was indicated above, the anhydrous composition according to the disclosure is in the form of a film comprising at least one film-forming polymer and at least one oxidizing agent.

In at least one embodiment of the present disclosure, the at least one oxidizing agent may be chosen from hydrogen peroxide; urea hydrogen peroxide; alkali metal, alkaline earth metal or ammonium percarbonates, perborates or periodates; alkali metal, alkaline earth metal or ammonium persulphates; alkali metal, alkaline earth metal or ammonium bromates; ferricyanides; copper or manganese salts; oxidizing quinones; and mixtures thereof.

In additional embodiments of the present disclosure, the oxidizing agent may be chosen from hydrogen peroxide or persalts. For example, the persalts may be chosen from chosen from alkaline earth metal or alkali metal persulphates, such as, for example, magnesium, sodium and potassium persulphates. In at least one embodiment, the persalts are chosen from sodium and potassium persulphates.

In at least one embodiment, the at least one oxidizing agent may be present in an amount ranging from 1 to 99.5% by weight relative to the total weight of the composition. For example, the oxidizing agent may be present in an amount ranging from 5 and 80% by weight relative to the weight of the composition.

The composition according to the present disclosure comprises at least one film-forming polymer which may be non-ionic, cationic, anionic, amphoteric.

The film-forming polymer of the composition according to the present disclosure may be such that, under the conditions of application of the composition to keratinous fibers, such as, for example, in the presence of an aqueous medium and upon, for instance, the massaging of the fibers and/or of homogenization, the composition may lose its cohesion and disintegrate.

In at least one embodiment of the present disclosure, the film-forming polymer may be chosen from polymers derived from vinylpyrrolidone, poly(vinyl alcohol), polyurethanes, polymers derived from caprolactam, vinyllactam or vinyl acetate, polymers derived from acrylamide, polysaccharides capable of forming a film in the dry state, such as cellulose derivatives, starches and derivatives, pullulan gum, gum arabic, pectins, alginates, carrageenans, galactomannans, agars, chitosans, chitins, polymers derived from hyaluronic acid, xanthan gum or karaya gum, proteins capable of forming a film in the dry state, such as gelatin, gluten, casein, zein, gliadin, hordein and their natural or synthetic derivatives, polymers derived from silicones, amphoteric or anionic polymers deriving from monomers comprising at least one carboxyl, sulpho or phosphoric functional group, acrylic copolymers of phosphorylcholine (Lipidure), anion-cation complexes of gum arabic/gelatin or gum arabic/chitosan type, or the collagen/GlycosAminoGlycan combination.

Non-limiting mention may also be made, as suitable cationic film-forming polymers, of the following polymers, generally having a number-average molecular weight ranging from 500 to 5,000,000:

(1) homopolymers or copolymers derived from acrylic or methacrylic esters or from acrylamides or methacrylamides and comprising at least one of the units of following formulae: 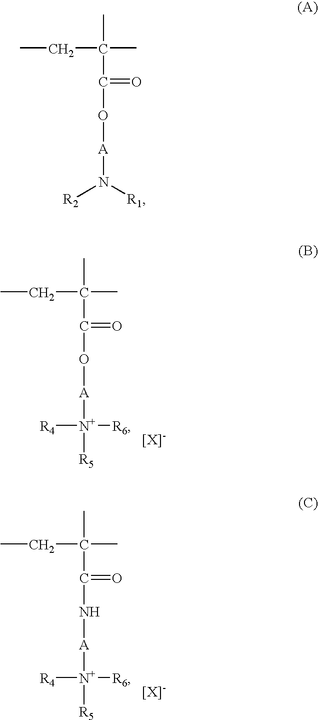
wherein:
R₁ and R₂, which may be identical or different, are chosen from hydrogen atoms and alkyl groups having from 1 to 6 carbon atoms;
R₃ is chosen from a hydrogen atom and a CH₃ group;
A is chosen from a linear or branched alkyl group comprising from 1 to 6 carbon atoms and a hydroxyalkyl group comprising from 1 to 4 carbon atoms;
R₄, R₅ and R₆, which are identical or different, are chosen from alkyl groups having from 1 to 18 carbon atoms and benzyl groups;
X is chosen from a methyl sulphate anion and a halide, such as chloride or bromide;

(2) quaternized guar gums;

(3) quaternary copolymers of vinylpyrrolidone and of vinylimidazole;

(4) chitosans or their salts;

the salts which can be used may be chosen from, for example, chitosan acetate, lactate, glutamate, gluconate and pyrrolidone-carboxylate.

The copolymers of the family (1) additionally comprise one or more units deriving from co-monomers which can be chosen from the family of the acrylamides, methacrylamides, diacetone acrylamides, acrylamides and methacrylamides substituted on the nitrogen by lower (C₁₋₄) alkyl groups, groups derived from acrylic or methacrylic acids or their esters, vinyllactams, such as vinylpyrrolidone or vinylcaprolactam, or vinyl esters.

Thus, non-limiting mention may be made, among these copolymers of the family (1), of:

copolymers of acrylamide and of dimethylaminoethyl methacrylate which are quaternized with dimethyl sulphate or with a methyl halide,

copolymers of acrylamide and of methacryloyloxy-ethyltrimethylammonium chloride, for example disclosed in European Patent Application EP-A-080 976,

copolymers of acrylamide and methacryloyloxy-ethyltrimethylammonium methyl sulphate,

quaternized or nonquaternized vinylpyrrolidone/dialkylaminoalkyl acrylate or methacrylate copolymers, such as the product sold under the “Gafquat®” name by ISP, such as, for example, “Gafquat® 734” or “Gafquat® 755”, or else the products known as “Copolymer® 845, 958 and 937”. These polymers are disclosed in detail in French Patent Nos. 2 077 143 and 2 393 573,

dimethylaminoethyl methacrylate/vinylcaprolactam/vinylpyrrolidone terpolymers, such as the product sold under the name Gaffix® VC 713 by ISP, and

vinylpyrrolidone/quaternized dimethylaminopropyl-methacrylamide copolymers, such as, for example, the product sold under the name “Gafquat® HS 100” by ISP.

Non-limiting mention may be made, among these compounds, of the chitosan having a degree of deacetylation of 90% by weight, the chitosan pyrrolidonecarboxylate sold under the name Kytamer® PC by Amerchol.

As regards the anionic film-forming polymers, the latter generally comprise at least one group derived from carboxylic, sulphonic or phosphoric acid and have a number-average molecular weight ranging from 500 to 5,000,000.

The carboxyl groups may be introduced by unsaturated mono- or dicarboxylic acid monomers, such as those corresponding to the formula:

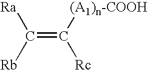

wherein:

n is an integer from 0 to 10;

A₁ is a methylene group optionally connected to the carbon atom of the unsaturated group or to the neighboring methylene group, when n is greater than 1, via a heteroatom, such as oxygen or sulphur;

Ra is chosen from a hydrogen atom and a phenyl or benzyl group;

Rb is chosen from a hydrogen atom, a C₁-C₄ alkyl group, such as, for example, methyl or ethyl, and a carboxyl group; and

Rc is chosen from a hydrogen atom, a lower alkyl group, a —CH₂—COOH group, a phenyl group, and a benzyl group.

In at least one embodiment of the present disclosure, the anionic film-forming polymers comprising carboxyl groups may be chosen from:

A) Homo- or copolymers of acrylic or methacrylic acid or their salts, such as, for example, the products sold under the names Versicol® E or K by Allied Colloid and Ultrahold® by BASF, copolymers of acrylic acid and of acrylamide, or sodium salts of polyhydroxycarboxylic acids.

B) Copolymers of acrylic or methacrylic acid with a monoethylenic monomer, such as ethylene, styrene, vinyl esters, or acrylic or methacrylic acid esters, which may also be grafted to a polyalkylene glycol, such as polyethylene glycol, and which are optionally crosslinked. Such polymers are disclosed, for example, in French Patent No. 1 222 944 and German Application No. 2 330 956, the copolymers of this type comprising, in their chain, an optionally N-alkylated and/or N-hydroxyalkylated acrylamide unit, such as are disclosed in Luxembourgian Patent Applications Nos. 75370 and 75371. Non-limiting mention may also be made of copolymers of acrylic acid and of C₁-C₄ alkyl methacrylate and terpolymers of vinylpyrrolidone, of acrylic acid and of C₁-C₂₀ alkyl methacrylate, for example lauryl methacrylate, such as that sold by ISP under the name Acrylidone® LM, and methacrylic acid/ethyl acrylate/tert-butyl acrylate terpolymers, such as the product sold under the name Luvimer® 100 P by BASF.

C) Copolymers derived from crotonic acid, such as those comprising, in their chain, vinyl acetate or propionate units, and optionally other monomers, such as allyl or methallyl esters, vinyl ether or vinyl ester of a saturated linear or branched carboxylic acid comprising a long hydrocarbon chain, such as those comprising at least 5 carbon atoms, it being possible for these polymers optionally to be grafted and crosslinked, or another monomer which is a vinyl, allyl or methallyl ester of an α- or β-cyclic carboxylic acid. Such polymers are disclosed, inter alia, in French Patents Nos. 1 222 944, 1 580 545, 2 265 782, 2 265 781, 1 564 110 and 2 439 798. A commercial product coming within this category is Resin 28-29-30, sold by National Starch.

D) Copolymers derived from monounsaturated C₄-C₈ carboxylic acids or anhydrides chosen from:

copolymers comprising (i) one or more maleic, fumaric or itaconic acids or anhydrides and (ii) at least one monomer chosen from vinyl esters, vinyl ethers, vinyl halides, phenylvinyl derivatives, or acrylic acid and its esters, the anhydride functional groups of these copolymers optionally being monoesterified or monoamidated. Such polymers are disclosed, for example, in U.S. Pat. Nos. 2,047,398; 2,723,248; and 2,102,113; and British Patent No. 839 805. Commercial products are include those sold under the names Gantrez® AN or ES by ISP.

copolymers comprising (i) one or more maleic, citraconic or itaconic anhydride units and (ii) one or more monomers chosen from allyl or methallyl esters optionally comprising one or more acrylamide, methacrylamide, α-olefin, acrylic or methacrylic ester, acrylic or methacrylic acid or vinylpyrrolidone groups in their chain,

the anhydride functional groups of these copolymers optionally being monoesterified or monoamidated.

These polymers are, for example, disclosed in French Patents Nos. 2 350 384 and 2 357 241, which belong to the present assignee.

E) Polyacrylamides comprising carboxylate groups.

F) Anionic polyurethanes, such as the product sold by BASF under the name Luviset PUR.

The polymers comprising sulphonic groups include polymers comprising vinylsulphonic, styrenesulphonic, naphthalenesulphonic or acrylamidoalkylsulphonic units.

These polymers may be chosen from, for example:

salts of polyvinylsulphonic acid having a molecular weight ranging from 1000 to 100,000 and the copolymers with an unsaturated comonomer, such as acrylic or methacrylic acids and their esters, acrylamide or its derivatives, vinyl ethers and vinylpyrrolidone.

salts of polystyrenesulphonic acid, such as the sodium salts, for example sold under the name Flexan® 130 by National Starch. These compounds are disclosed in French Patent No. FR 2 198 719.

salts of polyacrylamidosulphonic acids, such as those mentioned in U.S. Pat. No. 4,128,631 and, for example, polyacrylamidoethylpropanesulphonic acid.

According to the present disclosure, anionic film-forming polymers of grafted silicone type may also be used, such as those comprising a polysiloxane portion and a portion composed of a non-silicone organic chain, one of the two portions constituting the main chain of the polymer and the other being grafted onto the main chain. These polymers are disclosed, for example, in Patent Application Nos. EP-A-0 412 704, EP-A-0 412 707, EP-A-0 640 105 and WO 95/00578, EP-A-0 582 152 and WO 93/23009 and U.S. Pat. No. 4,693,935, U.S. Pat. No. 4,728,571 and U.S. Pat. No. 4,972,037.

Such polymers include, for example, the copolymers capable of being obtained by radical polymerization starting from the mixture of monomers comprising:

a) 50 to 90% by weight of tert-butyl acrylate;

b) 0 to 40% by weight of acrylic acid;

c) 5 to 40% by weight of silicone macromer of formula:

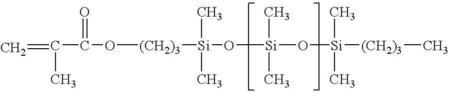

with v being a number ranging from 5 to 700; the percentages by weight being calculated with respect to the total weight of the monomers.

Other examples of grafted silicone polymers include polydimethylsiloxanes (PDMSs) onto which are grafted, via a connecting link of thiopropylene type, mixed polymer units of the poly((meth)acrylic acid) type and of the poly(alkyl(meth)acrylate) type and polydimethylsiloxanes (PDMSs) onto which are grafted, via a connecting link of thiopropylene type, polymer units of the poly(isobutyl(meth)acrylate) type.

Use may also be made, as film-forming polymers, of functionalized polyurethanes which may or may not comprise silicone portions.

Among polyurethanes that may be used according to the present disclosure, non-limiting mention may be made of those disclosed in Patent Nos. EP 0 751 162, EP 0 637 600, FR 2 743 297 and EP 0 648 485, and also Patent Nos. EP 0 656 021 or WO 94/03510 and EP 0 619 111.

According to at least one embodiment of the present disclosure, the anionic film-forming polymers may be chosen from acrylic acid copolymers, such as acrylic acid/ethylacrylate/N-(tert-butyl)acrylamide terpolymers, including those sold, for example, under the name Ultrahold® Strong by BASF, copolymers derived from crotonic acid, such as vinyl acetate/vinyl tert-butylbenzoate/crotonic acid terpolymers and crotonic acid/vinyl acetate/vinyl neododecanoate terpolymers, including those sold, for example, under the name Resin 28-29-30 by National Starch, polymers derived from maleic, fumaric or itaconic acids or anhydrides with vinyl esters, vinyl ethers, vinyl halides, phenylvinyl derivatives, or acrylic acid and its esters, such as monoesterified methyl vinyl ether/maleic anhydride copolymers, for example sold under the name Gantrez® by ISP, copolymers of methacrylic acid and of methylmethacrylate, sold under the name Eudragit® L by Rohm Pharma, copolymers of methacrylic acid and of ethyl acrylate, sold under the name Luvimer® MAEX or MAE by BASF, vinyl acetate/crotonic acid copolymers and vinyl acetate/crotonic acid copolymers grafted with polyethylene glycol, sold under the name Aristoflex® A by BASF, and the polyurethane Luviset PUR® sold by BASF.

In at least one embodiment of the present disclosure, the anionic film-forming polymers may be chosen from monoesterified methyl vinyl ether/maleic anhydride copolymers, sold under the name Gantrez® ES 425 by ISP, acrylic acid/ethyl acrylate/N-(tert-butyl)acrylamide terpolymers, sold under the name Ultrahold® Strong by BASF, copolymers of methacrylic acid and of methylmethacrylate, sold under the name Eudragit® L by Rohm Pharma, vinyl acetate/vinyl tert-butylbenzoate/crotonic acid terpolymers and crotonic acid/vinyl acetate/vinyl neododecanoate terpolymers, sold under the name Resin 28-29-30 by National Starch, copolymers of methacrylic acid and of ethyl acrylate, sold under the name Luvimer® MAEX or MAE by BASF, vinylpyrrolidone/acrylic acid/lauryl methacrylate terpolymers, sold under the name Acrylidone® LM by ISP, and the polyurethane Luviset PUR® sold by BASF.

Among the amphoteric film-forming polymers which can be used, non-limiting mention may be made of those comprising B and C units distributed randomly in the polymer chain, where B denotes a unit deriving from a monomer comprising at least one basic nitrogen atom and C denotes a unit deriving from an acidic monomer comprising at least one carboxyl or sulpho groups, or else B and C can denote groups deriving from zwitterionic carboxybetaine or sulphobetaine monomers;

B and C can also denote a cationic polymer chain comprising primary, secondary, tertiary or quaternary amine groups, wherein at least one of the amine groups carries a carboxyl or sulpho group connected via a hydrocarbon group, or else B and C form part of a chain of a polymer comprising an a,p-dicarboxyethylene unit, one of the carboxyl groups of which has been reacted with a polyamine comprising at least one primary or secondary amine groups.

Amphoteric film-forming polymers corresponding to the definition given above which may be used according to at least one embodiment of the present disclosure may be chosen from the following polymers:

(1) Polymers resulting from the copolymerization of a monomer derived from a vinyl compound carrying a carboxyl group, such as acrylic acid, methacrylic acid, maleic acid or α-chloroacrylic acid, and of a basic monomer derived from a substituted vinyl compound comprising at least one basic atom (such as, for example, an amino functional group), including dialkylaminoalkyl methacrylates and acrylates, dialkylaminoalkylmethacrylamides and dialkylamino-alkylacrylamides. Such compounds are disclosed in U.S. Pat. No. 3,836,537. Non-limiting mention may also be made of the sodium acrylate/acrylamido-propyltrimethylammonium chloride copolymer, sold under the name Polyquart KE 3033 by Henkel.

The vinyl compound can also be a dialkyldiallylammonium salt, such as diethyldiallyl-ammonium chloride. The copolymers of acrylic acid and of the latter monomer are provided under the names Merquat 280, Merquat 295 and Merquat Plus 3330 by Calgon.

(2) Polymers comprising units deriving:
a) from at least one monomer chosen from acrylamides or methacrylamides substituted on a nitrogen atom by an alkyl group,
b) from at least one acidic comonomer comprising one or more reactive carboxyl groups, and
c) from at least one basic comonomer, such as esters comprising primary, secondary, tertiary and quaternary amine substituents of acrylic and methacrylic acids and the quaternization product of dimethylaminoethyl methacrylate with dimethyl or diethyl sulphate.

The N-substituted acrylamides or methacrylamides which may be used according to at least one embodiment of the present disclosure include the compounds having alkyl groups comprising from 2 to 12 carbon atoms and may also include N-ethylacrylamide, N-(tert-butyl)acrylamide, N-(tert-octyl)acrylamide, N-octylacrylamide, N-decylacrylamide, N-dodecylacrylamide and the corresponding methacrylamides.

The acidic comonomers may also be chosen from acrylic, methacrylic, crotonic, itaconic, maleic or fumaric acids and the monoalkyl esters having 1 to 4 carbon atoms of maleic or fumaric acids or anhydrides.

Examples of co-monomers that may be used include aminoethyl, butylaminoethyl, N,N-dimethylaminoethyl and N-(tert-butyl)aminoethyl methacrylates.

In some embodiments of the present disclosure, use may be made, for example, of the copolymers having the CTFA name (4th Ed., 1991) of Octylacrylamide/acrylate/butylaminoethyl methacrylate copolymer, such as the products sold under the name Amphomer® or Lovocryl® 47 by National Starch.

(3) Partially or completely acylated and crosslinked polyaminoamides deriving from polyaminoamides of general formula: [symbol]
CO—R₁₀—CO-Z [symbol] (III)

wherein R₁₀ is chosen from a divalent group derived from a saturated dicarboxylic acid, an aliphatic mono- or dicarboxylic acid comprising an ethylenic double bond, an ester of a lower alkanol having 1 to 6 carbon atoms of these acids, and a group deriving from the reaction of any one of the said acids with a bisprimary or bissecondary amine, and Z is chosen from a group derived from a bisprimary, monosecondary or bissecondary polyalkylenepolyamine and may represent, for example:
a) in proportions of 60 to 100 mol %, the group 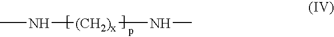
where x=2 and p=2 or 3, or where x=3 and p=2,
this group derived from diethylenetriamine, triethylenetetraamine or dipropylenetriamine;
b) in proportions of 0 to 40 mol %, the above group (IV), wherein x=2 and p=1 and which derives from ethylenediamine, or the group deriving from piperazine: 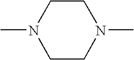
c) in proportions of 0 to 20 mol %, the —NH—(CH₂)₆—NH— group deriving from hexamethylenediamine,

these polyaminoamides being crosslinked by an addition reaction of a bifunctional crosslinking agent chosen from epihalohydrins, diepoxides, dianhydrides and bisunsaturated derivatives, using from 0.025 to 0.35 mol of crosslinking agent per amine group of the polyaminoamide, and acylated by the action of acrylic acid, chloroacetic acid or an alkane sultone, or their salts.

The saturated carboxylic acids, for example, may be chosen from acids having 6 to 10 carbon atoms, such as adipic, 2,2,4-trimethyladipic, 2,4,4-trimethyladipic or terephthalic acids, or acids comprising an ethylenic double bond, such as, for example, acrylic, methacrylic or itaconic acids.

The alkane sultones used in the acylation may be chosen from, for example, propane and butane sultone and the salts of the acylating agents may include, for example, the sodium or potassium salts.

(4) Polymers comprising zwitterionic units of formula:

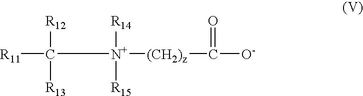

wherein:

R¹¹ is a polymerizable unsaturated group, such as an acrylate, methacrylate, acrylamide or methacrylamide group,

y and z represent an integer from 1 to 3,

R₁₂ and R₁₃ are independently chosen from hydrogen atoms and from methyl, ethyl and propyl groups, and

R₁₄ and R₁₅ are independently chosen from hydrogen atoms and alkyl groups so that the sum of the carbon atoms in R₁₄ and R₁₅ does not exceed 10.

The polymers comprising such units can also comprise units derived from non-zwitterionic monomers, such as dimethylaminoethyl acrylate, dimethylaminoethyl methacrylate, diethylaminoethyl acrylate, diethylamino-ethyl methacrylate, alkyl acrylates, alkyl methacrylates, acrylamides, methacrylamides or vinyl acetate.

Mention may be made, by way of example, of butylmethacrylate/N,N-dimethylcarboxyamionethyl methacrylate copolymers.

(5) Polymers derived from chitosan comprising monomeric units corresponding to the following

formulae:

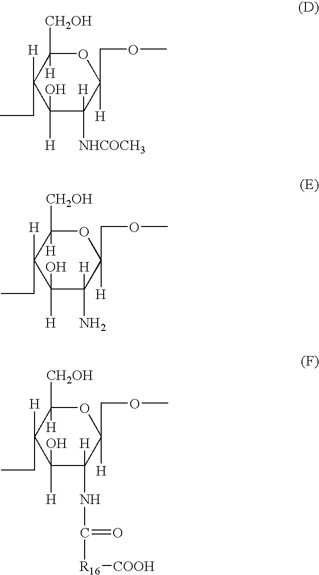

The unit (D) being present in proportions ranging from 0 to 30%, the unit (E) in proportions ranging from 5 to 50% and the unit (F) in proportions ranging from 30 to 90%, it being understood that, in this unit (F), R₁₆ is chosen from groups of formula:

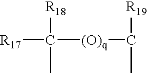

wherein:

if q=0, then R₁₇, R₁₈ and R₁₉, which are identical or different, are chosen from hydrogen atoms, methyl, hydroxyl, acetoxy and amino residues, monoalkylamino residues and a dialkylamino residues which are optionally interrupted by at least one nitrogen atom and/or optionally substituted by at least one amino, hydroxyl, carboxyl, alkylthio or sulpho group, and an alkylthio residue, the alkyl group of which carries an amino residue, at least one of the R₁₇, R₁₈ and R₁₉ groups being, in this case, a hydrogen atom;

or, if q=1, then R₁₇, R₁₈ and R₁₉ each represent a hydrogen atom, and the salts formed by these compounds with bases or acids.

(6) Polymers corresponding to the general formula (VI) are, for example, disclosed in French Patent No. 1 400 366 and comprise the repeating unit below:

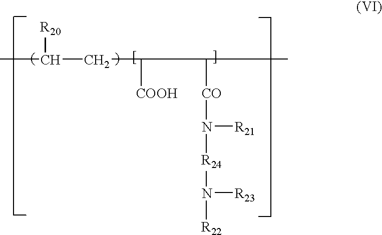

wherein:

R₂₀ is chosen from a hydrogen atom, a CH₃O group, a CH₃CH₂O group, and a phenyl group,

R₂₁ is chosen from a hydrogen atom and lower alkyl groups, such as methyl or ethyl,

R₂₂ is chosen from a hydrogen atom and lower C₁-C₆ alkyl groups, such as methyl or ethyl,

R₂₄ is chosen from —CH₂—CH₂—, —CH₂—CH₂—CH₂— and —CH₂—CH(CH₃)— groups, and

R₂₃ is chosen from lower C₁-C₆ alkyl groups, such as methyl or ethyl, and a group corresponding to the formula: —R₂₄—N(R₂₂)₂, wherein R₂₄ is chosen from —CH₂—CH₂—, —CH₂—CH₂—CH₂— and —CH₂—CH(CH₃)— groups and R₂₂ has the meanings mentioned above.

(7) Polymers derived from the N-carboxyalkylation of chitosan, such as N-(carboxymethyl)chitosan or N-(carboxybutyl)chitosan.

(8) Amphoteric polymers of the -D-X-D-X- type chosen from:
a) the polymers obtained by the reaction of chloroacetic acid or sodium choloroacetate with compounds comprising at least one unit of formula -D-X-D-X-D- (VII), where D denotes a group

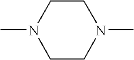

and X denotes the symbol E or E′, with E or E′, which are identical or different, denoting a divalent group which is a straight- or branched-chain alkylene group comprising up to 7 carbon atoms in the main chain, which is optionally substituted by hydroxyl groups and which can additionally comprise oxygen, nitrogen or sulphur atoms, 1 to 3 aromatic and/or heterocyclic rings; the oxygen, nitrogen and sulphur atoms being present in the form of ether, thioether, sulphoxide, sulphone, sulphonium, alkylamine, alkenylamine, hydroxyl, benzylamine, amine oxide, quaternary ammonium, amide, imide, alcohol, ester and/or urethane groups.
b) The polymers of formula: -D-X-D-X- (VII′)

where D denotes a group

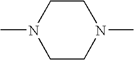

and X denotes the symbol E or E′ and at least once E′; E having the meaning indicated above and E′ being a divalent group which is a straight- or branched-chain alkylene group having up to 7 carbon atoms in the main chain, which is optionally substituted by at least one hydroxyl group and which comprises one or more nitrogen atoms, the nitrogen atom being substituted by an alkyl chain optionally interrupted by an oxygen atom and comprising at least one carboxyl functional group or at least one hydroxyl functional group and betainized by reaction with chloroacetic acid or sodium chloracetate.

(9) C₁-C₅ alkyl vinyl ether/maleic anhydride copolymers partially modified by semiamidation with an N,N-dialkyl aminoalkylamine, such as N,N-dimethylaminopropylamine, or by semiesterification with an N,N-dialkylamino-alkanol. These copolymers can also comprise other vinyl comonomers, such as vinyl caprolactam.

In at least one embodiment of the present disclosure, the amphoteric film-forming polymers are those of the family (3), such as the copolymers having the CTFA name of Octylacrylamide/acrylate/butylaminoethyl methacrylate copolymer, such as the products sold under the names Amphomer®, Amphomer® LV 71 or Lovocryl® 47 by National Starch, and those of the family (4), such as butylmethacrylate/N,N-dimethylcarboxyaminoethyl methacrylate copolymers.

Among the non-ionic film-forming polymers which can be used according to the present disclosure, non-limiting mention may be made of those chosen, for example, from:

vinyl acetate homopolymers;

copolymers of vinyl acetate and of acrylic ester;

copolymers of vinyl acetate and of ethylene;

copolymers of vinyl acetate and of maleic ester, for example dibutylmaleate;

copolymers of acrylic esters, such as, for example, copolymers of alkyl acrylates and of alkyl methacrylates, such as the products provided by Rohm & Haas under the names Primal® AC-261 K and Eudragit® NE 30 D, by BASF under the name 8845 or by Hoechst under the name Appretan® N9212;

copolymers of acrylonitrile and of a non-ionic monomer, for example chosen from butadiene and alkyl(meth)acrylates; non-limiting mention may be made of the products provided under the name CJ 0601 B by Rohm & Haas;

styrene homopolymers;

copolymers of styrene and of alkyl (meth)acrylate, such as the products Mowilith® LDM 6911, Mowilith® DM 611 and Mowilith® LDM 6070 provided by Hoechst or the products Rhodopas® SD 215 and Rhodopas® DS 910 provided by Rhodia Chimie;

copolymers of styrene, of alkyl methacrylate and of alkyl acrylate;

non-ionic polyurethanes;

copolymers of styrene and of butadiene;

copolymers of styrene, of butadiene and of vinylpyridine;

copolymers of alkyl acrylate and of urethane;

polyamides;

vinyllactam homopolymers and copolymers.

In at least one embodiment of the present disclosure, the alkyl groups of the non-ionic polymers mentioned above have from 1 to 6 carbon atoms.

According to the present disclosure, the film-forming polymers may include non-ionic polymers, such as, for example, non-ionic polymers comprising vinyllactam units. They are disclosed in U.S. Pat. No. 3,770,683, U.S. Pat. No. 3,929,735, U.S. Pat. No. 4,521,504, U.S. Pat. No. 5,158,762 and U.S. Pat. No. 5,506,315 and in International Patent Application Nos. WO 94/121148, WO 96/06592 and WO 96/10593. They can be provided in a pulverulent form or in the solution or suspension form.

The homopolymers or copolymers comprising vinyllactam units comprise units of formula:

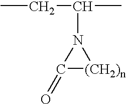

wherein n is independently 3, 4 or 5.

The number-average molecular weight of the polymers comprising vinyllactam units is generally greater than 5000, such as from 10,000 to 1,000,000, or from 10,000 to 100,000.

Use may also be made, as a film-forming polymer in the present disclosure, of polyvinylpyrrolidones, such as those sold under the name Luviskol® K30 by BASF; polyvinylcaprolactams, such as those sold under the name Luviskol® PLUS by BASF; poly(vinylpyrrolidone/vinyl acetate) copolymers, such as those sold under the name PVPVA® S630L by ISP or Luviskol® VA 73, VA 64, VA 55, VA 37 and VA 28 by BASF; and poly(vinylpyrrolidone/vinyl acetate/vinylpropionate)terpolymers, such as, for example, those sold under the name Luviskol® VAP 343 by BASF.

In at least one embodiment of the present disclosure, the film-forming polymers may be chosen from water-soluble or water-dispersible film-forming polymers. For example, in at least one embodiment of the present disclosure, the film-forming polymers are chosen from water-soluble film-forming polymers.

The amount of film-forming polymer present in the composition may range, for example, from 0.5 to 97% by weight relative to the total weight of the composition. For example, the amount of film-forming polymer may range from 5 to 90% by weight relative to the total weight of the composition.

The composition according to the present disclosure can additionally comprise at least one plasticizing agent.

The plasticizing agents, if they are present, may be chosen from the compounds conventionally used in this field.

In at least one embodiment of the present disclosure, the plasticizing agent may be chosen from glycerol, sorbitol, mono- and/or disaccharides, dipropylene glycol, butylene glycol, pentylene glycol or polyethylene glycol, such as, for example, PEG 400 and PEG 4000.

If they are present in the composition, the amount of plasticizing agent present in the composition may range, for example, from 0.05 to 20% by weight relative to the total weight of the composition.

The composition may also comprise at least one alkaline or acidic pH regulating agent.

Mention may be made, among the alkaline agents, by way of examples, of aqueous ammonia solution, ammonium salts, alkaline carbonates, alkanolamines, such as mono-, di- and triethanolamines, and their derivatives, hydroxyalkylamines and ethylenediamines which are oxyethylenated and/or oxypropylenated, sodium hydroxide, potassium hydroxide and the compounds with the following formula:

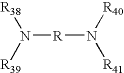

wherein:

R is a propylene residue optionally substituted by a hydroxyl group or a C₁-C₄ alkyl radical, and

R₃₈, R₃₉, R₄₀ and R₄₁, which are identical or different, are chosen from hydrogen atoms, and C₁-C₄ alkyl, and C₁-C₄ hydroxyalkyl radicals.

Among the acidic agents, mention may be made, for example, of inorganic or organic acids, such as hydrochloric acid, orthophosphoric acid, sulphuric acid, carboxylic acids, such as acetic acid, tartaric acid, citric acid or lactic acid, sulphonic acids or their mixtures.

The amount of alkaline and/or acidic agent is such that the pH of the composition brought into contact with water may range from 3 to 11, such as, for example, from 7to 11.

The composition according to the present disclosure can also comprise the conventional additives encountered in formulations intended for the dyeing of keratinous fibers, provided that the composition according to the present disclosure retains the appearance of a film and that its ability to disintegrate in the presence of an aqueous medium is not detrimentally affected.

Mention may be made, by way of example, of non-ionic, anionic, cationic or amphoteric surface-active agents; penetrating agents; sequestering agents; fragrances; buffers; dispersing agents; conditioning agents, such as, for example, cations, cationic polymers or volatile or nonvolatile and modified or unmodified silicones; ceramides; preservatives; stabilizing agents; opacifying agents, and the like.

The additives may be present in an amount ranging from 0 to 30% by weight relative to the total weight of the composition.

The composition according to the present disclosure may also comprise at least one direct dye.

The at least one direct dye may be an ionic or non-ionic entity. In at least one embodiment of the present disclosure, the at least one direct dye may be chosen from cationic or non-ionic entities.

Mention may be made, as non-limiting examples, of nitrobenzene dyes, azo, azomethine, methine, tetraazapentamethine, anthraquinone, naphthoquinone, benzoquinone, phenothiazine, indigoid, xanthene, phenanthridine or phthalocyanine dyes, those derived from triarylmethane and natural dyes, alone or as mixtures.

The at least one direct dye can be chosen from, for example, red and orange, yellow and yellow-green, blue and purple nitrobenzene dyes.

Non-limiting mention may be made, among the azo direct dyes which can be used according to the present disclosure, of the cationic azo dyes disclosed in Patent Application Nos. WO 95/15144, WO 95/01772, EP 714 954, FR 2 822 696, FR 2 825 702, FR 2 825 625, FR 2 822 698, FR 2 822 693, FR 2 822 694, FR 2 829 926, FR 2 807 650, WO 02/078660, WO 02/100834, WO 02/100369 and FR 2 844 269.

Non-limiting mention may also be made, among azo direct dyes, of the following dyes, described in the Color Index International, 3rd edition: Disperse Red 17, Acid Yellow 9, Acid Black 1, Basic Red 22, Basic Red 76, Basic Yellow 57, Basic Brown 16, Acid Yellow 36, Acid Orange 7, Acid Red 33, Acid Red 35, Basic Brown 17, Acid Yellow 23, Acid Orange 24 or Disperse Black 9.

Non-limiting mention may also be made of 1-(4′-aminodi-phenylazo)-2-methyl-4-bis[(β-hydroxyethyl)amino]benzene and 4-hydroxy-3-(2-methoxyphenylazo)-1-naphthalenesulphonic acid.

Non-limiting mention may be made, among quinone dyes, of the following dyes: Disperse Red 15, Solvent Violet 13, Acid Violet 43, Disperse Violet 1, Disperse Violet 4, Disperse Blue 1, Disperse Violet 8, Disperse Blue 3, Disperse Red 11, Acid Blue 62, Disperse Blue 7, Basic Blue 22, Disperse Violet 15, Basic Blue 99, and the like.

Non-limiting mention may be made, among azine dyes, of Basic Blue 17 or Basic Red 2.

Non-limiting mention may also be made, among cationic methine direct dyes, of Basic Red 14, Basic Yellow 13 and Basic Yellow 29.

Non-limiting mention may be made, among triarylmethane dyes which can be used according to the present disclosure, of the following compounds: Basic Green 1, Acid Blue 9, Basic Violet 3, Basic Violet 14, Basic Blue 7, Acid Violet 49, Basic Blue 26 or Acid Blue 7.

The composition can also comprise natural direct dyes, such as lawsone, juglone, alizarin, purpurin, carminic acid, kermesic acid, purpurogallin, protocatechualdehyde, indigo, isatin, curcumin, spinulosin or apigenidin.

In at least one embodiment, if at least one direct dye is present, the total amount may range from 0 to 3% by weight relative to the total weight of the composition.

According to an embodiment of the present disclosure, the composition is provided in the form of a film, the thickness of which may range from 10 to 2000 μm, such as, for example, from 20 to 500 μm.

In accordance with other embodiments of the present disclosure, the composition may be deposited on a water-insoluble support which is inert with regard to the composition and with regard to the composition when the latter is brought into contact with an aqueous medium.

In at least one embodiment, the water-insoluble support may be chosen from polyurethanes, thermoplastic elastomers of the styrene-butadiene-styrene, styrene-ethylene-butadiene-styrene, ethylene-vinyl acetate or coether ester type, polyethylenes, polypropylenes, silicones, metal sheets or films, such as aluminium, composite sheets or films comprising polytetrafluoroethylene, polyamide copolymers comprising polyether blocks, poly(vinylidene chloride), nylon, elastomers of isobutylene-styrene or styrene-isoprene type, or materials.

Such supports are sold, for example, under the trade names: Baydur®, Daltoflex®, Uroflex®, Hyperlast®, Inspire®, Desmopan®, Estane®, Lastane®, Texin®, Cariflex®, Kraton®, Solprene®, Elvax®, Escorene®, Optene®, Amitel®, Hytrel® or Riteflex®.

Alternatively, the support can be in the form of a non-woven, made of, for example, cellulose, viscose, cotton or synthetic fibers.

The nature and the form of the support will be appropriately chosen in order to allow the user to bring the film into contact with the surface to be treated and to allow the massaging thereof on the surface without risk to the latter and with maximum comfort.

The thickness of the support may range from 0.01 mm to 2 mm, such as, for example, from 0.02 to 0.2 mm.

The composition according to the present disclosure can be obtained by applying, to a support, a precursor composition comprising, in an appropriate solvent, a mixture comprising at least one oxidizing agent and at least one film-forming polymer and then the solvent is evaporated.

It should be noted that the contents of at least one oxidizing agent and at least one film-forming polymer in the precursor composition are such that, once the solvent has evaporated, the concentration ranges of the composition which are described in detail above are adhered to.

The precursor composition can also provide at least one acidic or alkaline pH regulating agent in proportions such that the conditions described for the final composition are adhered to.

Finally, the composition can comprise additives which are conventional in the field and reference may be made to the list indicated above. Here again, their content in the precursor is such that, once the solvent has evaporated, the concentration ranges in the composition which are described in detail above are adhered to.

As indicated above, the precursor composition comprises at least one solvent. The latter is chosen so that the compounds present in the precursor composition are soluble or dispersed therein.

In at least one embodiment, the boiling point of the solvent is less than or equal to 200° C.

Mention may be made, as examples of solvents which can be used, for example, of water, ethanol, acetone, isopropanol, ethyl acetate, dichloromethane, ethyl ether and the like. When the precursor composition comprises a basifying agent, the solvent may be chosen from a solvent other than water.

The solvent content is such that it is compatible with easy spreading of the precursor composition, making it possible to control the thickness thereof.

In accordance with at least one embodiment of the present disclosure, the solvent is present in an amount ranging from 10 to 95% by weight relative to the total weight of the precursor composition.

The composition can be obtained by mixing the various compounds and by then applying the precursor composition thus obtained to an appropriate support, such as, for example, a smooth and horizontal support of heating or non-heating bench or plate type.

In at least one embodiment, the composition may be deposited directly on the support with which the composition is intended to be used, if such an alternative form is chosen.

The composition may be deposited conventionally, such as with a device which makes it possible to obtain a substantially uniform film thickness.

After deposition of the composition, the solvent is evaporated conventionally, such as in an oven.

Another aspect of the present disclosure relates to a process for the treatment of keratinous fibers, such as human keratinous fibers, in which the fibers are brought into contact with the composition according to the present disclosure in the presence of an aqueous medium.

The fibers treated can be dry or wet. In the latter case, the water present on the fibers can constitute, in all or in part, the aqueous medium mentioned above.

The aqueous medium, in addition to water, can optionally comprise at least one different adjuvant conventionally used in the field of the treatment of keratinous fibers.

Thus, in the case where the composition is intended for the dyeing of keratinous fibers, the aqueous medium comprises at least one oxidation dye and/or at least one direct dye.

The oxidation dye may be chosen from oxidation bases, optionally in combination with at least one coupler.

The oxidation bases conventionally used for oxidation dyeing are employed and can be chosen, for example, from para-phenylenediamines, bisphenylalkylenediamines, para-aminophenols, ortho-aminophenols and heterocyclic bases, and their addition salts with an acid or with an alkaline agent.

The coupler or couplers may be chosen from the compounds conventionally used in oxidation dyeing, and among which non-limiting mention may be made, for example, of meta-phenylenediamines, meta-aminophenols, meta-diphenols and heterocyclic couplers, and their addition salts with an acid or with an alkaline agent.

The addition salts with an acid may be chosen, for example, from hydrochlorides, hydrobromides, sulphates, citrates, succinates, tartrates, tosylates, benzenesulphonates, lactates and acetates.

In at least one embodiment, the oxidation base or bases are present in an amount ranging from 0.005 to 6% by weight relative to the total weight of the composition applied to the fibers.

When they are present, the coupler or couplers are present in an amount ranging from 0.005 to 5% by weight relative to the total weight of the composition applied to the fibers.

The direct dyes which can be used are chosen from the usual compounds in the field and can be ionic or non-ionic entities.

Mention may be made, as non-limiting examples of direct dyes, of nitrobenzene, azo, azomethine, methine, tetraazapentamethine, anthraquinone, naphthoquinone, benzoquinone, phenothiazine, indigoid, xanthene, phenanthridine or phthalocyanine dyes, those derived from triarylmethane and natural dyes, alone or as mixtures.

The amount of additional direct dyes may range, for example, from 0.005 to 6% by weightrelative to the total weight of the composition applied to the fibers.

In embodiments where the composition according to the present disclosure is intended to be applied in the field of bleaching or in the fixing stage of a permanent deformation process, the aqueous medium can comprise at least one additional oxidizing agent and/or at least one basifying agent chosen from the compounds mentioned above.

Whatever the field of use of the composition, the aqueous medium can likewise comprise pH regulating agents, such as, for example, alkaline or acidic agents. Reference may be made to the description with regard to the nature of these agents.

The respective amounts of the pH regulating agents can be such that the pH of the composition and of the aqueous medium ranges from 3 to 11, such as, for example, from 7 to 11.

Whatever the use of the composition (e.g., dyeing, bleaching, permanent deformation), the aqueous medium can comprise at least one adjuvant chosen from non-ionic, anionic, cationic and amphoteric surfactants; cationic and amphoteric substantive polymers; anionic, cationic, non-ionic, amphoteric and zwitterionic thickening polymers; inorganic thickening agents; antioxidants; α-oxocarboxylic acids (for example, oxalic acid, glyoxalic acid, pyruvic acid and α-ketoglutaric acid); penetrating agents; fragrances; buffers; basic amino acids, such as arginine; dispersing agents; peptizing agents; conditioning agents, such as, for example, cations and volatile or non-volatile and modified or unmodified silicones; ceramides; vitamins and provitamins; preservatives; stabilizing agents; opacifying or mattifying agents, such as titanium dioxide; inorganic fillers, such as clays or silicas, including pyrogenic silicas, with a hydrophilic or hydrophobic nature; lubricants, such as stearates; agents for controlling the release of oxygen, such as magnesium carbonate or oxide; sunscreens, and the like.

The adjuvants mentioned above can be present in an amount ranging from 0 to 20% by weight for each of them relative to the total weight of the composition of the aqueous medium.

The aqueous medium can be liquid.

The amount of aqueous medium may be such that it makes possible the release of the dye present in the composition by disintegration of the film.

According to one embodiment of the present disclosure, the composition and the aqueous medium may be applied successively or else the composition according to the present disclosure is applied to the wet fibers, optionally followed by application of an additional aqueous medium. Once these operations have been carried out, the fibers may be massaged in order to promote the disintegration of the film and the distribution of the dye thus released over the whole of the fibers to be treated.

According to another embodiment, the composition according to the present disclosure may be mixed with the aqueous medium prior to the application to the dry or wet keratinous fibers.

Whatever the alternative form selected, the composition may be left to stand on the fibers until the desired effect (e.g., dyeing, bleaching, fixing) is obtained.

The leave-in time may vary from 1 to 60 minutes, such as, for example, from 5 to 45 minutes.

The application temperature may range, for example, from 15° C. to 220° C., such as, for example, in the region of ambient temperature.

Once the leave-in time has passed, the fibers may be rinsed, optionally washed with shampoo and then rinsed again, before being dried or left to dry.

As was mentioned previously, the composition according to the present disclosure can be used in the field of dyeing.

It can likewise be used for bleaching keratinous fibers.

In the case where moderate bleaching is desired, such as, for example, a difference of the order of two tones, the composition in the form of a film according to the present disclosure may comprise a peroxide as an oxidizing agent. In at least one embodiment, the pH regulating agent is introduced into the aqueous medium.

In the case where a strong (e.g., difference of 4 tones) to intense (e.g., difference of 6 tones) degree of bleaching is desired, the composition according to the present disclosure may comprise a persalt as oxidizing agent. In at least one embodiment, the composition in the form of a film according to the present disclosure may additionally comprise at least one basifying agent. The aqueous medium may also comprise at least one oxidizing agent, such as, for example, peroxide. Furthermore, the aqueous medium may comprise at least one basifying agent.

The lightening of the hair may be evaluated by the “tone height”, which characterizes the degree of a level of lightening. The notion of “tone” is based on the classification of the natural shades, one tone separating each shade from that which immediately follows it or precedes it. This definition and the classification of the natural shades are well known to professionals in hair styling and are published in the work “Sciences des traitements capillaires [Hair Treatment Sciences] by Charles Zviak, 1988, published by Masson, pp. 215 and 278. The tone heights range from 1 (black) to 10 (light light blond), one unit corresponding to one tone; the higher the figure, the lighter the shade.

In the case where the composition according to the present disclosure is intended to be employed in a process for the permanent deformation of keratinous fibers, the composition may be applied after the reduction stage (that is say, a stage during which the disulphide bridges of the fibers are opened).

The reduction stage may be carried out by applying a composition comprising at least one reducing agent chosen from, for example, sulphur-comprising compounds and also compounds exhibiting at least one thiol, sulphite or sulphinic functional group, and the salt forms thereof.

In at least one embodiment, the reducing agent, if it is present, may be chosen from cysteine, thioglycolic acid or its salts, thiolactic acid and its salts, and cysteamine, and mixtures thereof.

The amount of reducing agent in the reducing composition may range from 0.01 to 10% by weight relative to the weight of this composition, such as, for example, from 0.1 and 5% by weight.

The reducing composition can additionally comprise conventional adjuvants and reference may be made to the lists given above.

The reducing composition may be applied to the dry or wet keratinous fibers, the latter being shaped before, during or after the application of the reducing composition.

The leave-in time of the composition can range from a few seconds to 30 minutes, such as, for example, from 3 to 15 minutes.

The temperature at which the reducing composition is left to act is can range from 15° C. to 220° C., such as, for example, from 15° C. to 80° C., or in the region of ambient temperature.

Once the leave-in time has passed, the fibers may be rinsed in order to remove the excess reducing composition.

The composition according to the present disclosure is then subsequently applied, as was described above.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding the numerical ranges and parameters setting forth the broad scope of the invention are approximations, the numerical values set forth in the specific examples are reported as precisely as possible. Any numerical value, however, inherently contains certain errors necessarily resulting from the standard deviation found in its respective testing measurement.

The examples that follow are intended to illustrate the invention without, however, being limiting in nature.

In the examples, the percentages are expressed as weight percentages of active material.

### EXAMPLE 1

| | | |
|---|---|---|
| Hydroxypropylmethylcellulose | 10 | g |
| Hydrogen peroxide | 9 | g |
| Water | 36.5 | g |

All the ingredients were mixed with stirring.

The solution was deposited on silicone paper in a thickness of the order of 850 μm and then dried.

After drying, the film obtained was cut into the form of strips with a width of 40 mm and a length of 80 mm.

40 g of the following dyeing composition were spread:

| Oleyl alcohol polyglycerolated with 2 mol of glycerol | 2 | g |
|---|---|---|
| Oleyl alcohol polyglycerolated with 4 mol of glycerol | 2.84 | g AM |
| (78% AM) | | |
| Oleic acid | 1.5 | g |
| Oleylamine comprising 2 mol of ethyleneoxide, | 3.5 | g |
| sold under the trade name Ethomeen O12 by Akzo | | |
| Diethylaminopropyl laurylaminosuccinamate, sodium | 1.5 | g AM |
| salt, comprising 55% AM | | |
| Oleyl alcohol | 2.5 | g |
| Oleic acid diethanolamide | 6.0 | g |
| Propylene glycol | 1.75 | g |
| Ethyl alcohol | 3.5 | g |
| Dipropylene glycol | 0.25 | g |
| Propylene glycol monomethyl ether | 4.5 | g |
| Sodium metabisulphite as a 35% AM aqueous solution | 0.227 | g AM |
| Ammonium acetate | 0.4 | g |
| 1-Hydroxy-3-aminobenzene | 0.022 | g |
| 1,3-Dihydroxybenzene | 0.22 | g |
| 1,3-Bis(4-aminophenyl)(2-hydroxyethylamino)-2- | 0.025 | g |
| propanol tetrahydrochloride | | |
| 2-Methyl-1,3-dihydroxybenzene | 0.068 | g |
| 1-Methyl-2,5-diaminobenzene | 0.162 | g |
| Antioxidant, sequestering agent | q.s. | |
| Fragrance, preservative | q.s. | |
| Aqueous ammonia solution comprising 20% of NH₃ | 5 | g |

Immediately afterwards, two strips obtained above were applied and were spread over the hair by massaging.

The hair was left standing at ambient temperature for 30 minutes.

After rinsing, shampooing and drying, the hair was colored in a blond shade.

### EXAMPLE 2

| | | |
|---|---|---|
| Hydroxypropylcellulose | 10 | g |
| Potassium persulphate | 30 | g |
| Ethanol | 27.5 | g |

All the ingredients were mixed with stirring.

The dispersion was deposited on silicone paper in a thickness of the order of 1 mm and then dried at ambient temperature.

After drying, the film obtained was cut into the form of strips with a width of 40 mm and a length of 80 mm.

2 g of a bleaching cream, obtained by mixing 0.8 g of an anhydrous composition of formula A below with 1.2 g of a 6% by weight aqueous hydrogen peroxide composition, were applied to a lock of dark brown hair.

Formula A:

| | | |
|---|---|---|
| Sodium disilicate | 15 | g |
| Sodium metasilicate | 3 | g |
| Ammonium chloride | 4.2 | g |
| EDTA | 0.2 | g |
| Serad FX 1100 | 2 | g |
| Primogel | 2 | g |
| Sodium lauryl sulphate | 4 | g |
| Sodium stearate | 2 | g |
| Magnesium stearate | 2 | g |
| Amorphous silica | 1 | g |
| Titanium oxide | 1 | g |
| Mineral oil | 1 | g |
| Beeswax | 1 | g |
| Isopropyl myristate | 21.6 | g |

Immediately afterwards, two strips obtained above were applied to the lock and were spread over the lock by massaging. The hair was left standing at ambient temperature for 40 minutes. After rinsing and drying, a blond lock was obtained.

What is claimed is:

1. A process for the treatment of keratinous fibers, comprising:
applying to said fibers an anhydrous composition in the form of a film, wherein the anhydrous composition comprises at least one film-forming polymer and at least one oxidizing agent, and
then, bringing said fibers into contact with an aqueous medium.

2. The process according to claim 1, wherein the at least one oxidizing agent is chosen from hydrogen peroxide; urea hydrogen peroxide; alkali metal, alkaline earth metal or ammonium percarbonates, perborates or periodates; alkali metal, alkaline earth metal or ammonium persulphates; alkali metal, alkaline earth metal or ammonium bromates; ferricyanides; copper or manganese salts; oxidizing quinones; and mixtures thereof.

3. The process according to claim 1, wherein the at least one oxidizing agent is present in the anhydrous composition in an amount ranging from 1 to 99.5% by weight relative to the total weight of the anhydrous composition.

4. The process according to claim 3, wherein the at least one oxidizing agent is present in the anhydrous composition in an amount ranging from 5 to 80% by weight relative to the total weight of the anhydrous composition.

5. The process according to claim 1, wherein the at least one film-forming polymer is chosen from polymers derived from: vinylpyrrolidone; poly(vinyl alcohol); polyurethanes; caprolactam; vinyllactam; vinyl acetate; acrylamide; polysaccharides capable of forming a film in the dry state; proteins capable of forming a film in the dry state; silicones, amphoteric and anionic polymers derived from monomers comprising at least one carboxyl, sulpho or phosphoric functional group; acrylic copolymers of phosphorylcholine; anion-cation complexes of gum arabic/gelatin or gum arabic/chitosan type; and a collagen/GlycosAminoGlycan combination.

6. The process according to claim 5, wherein the polysaccharides capable of forming a film in the dry state are chosen from cellulose derivatives, starches and derivatives, pullulan gum, gum arabic, pectins, alginates, carrageenans, galactomannans, agars, chitosans, chitins, hyaluronic acid, xanthan gum, and karaya gum.

7. The process according to claim 5, wherein the proteins capable of forming a film in the dry state are chosen from gelatin, gluten, casein, zein, gliadin, hordein and their natural or synthetic derivatives

8. The process according to claim 1, wherein the at least one film-forming polymer is water-soluble or water-dispersible.

9. The process according to claim 8, wherein the at least one film-forming polymer is water-soluble.

10. The process according to claim 1, wherein the at least one film-forming polymer is present in the anhydrous composition in an amount ranging from 0.1 to 97% by weight relative to the total weight of the anhydrous composition.

11. The process according to claim 1, wherein the anhydrous composition further comprises at least one plasticizing agent.

12. The process according to claim 11, wherein the at least one plasticizing agent is chosen from glycerol, sorbitol, monosaccharides, disaccharides, dipropylene glycol, butylene glycol, pentylene glycol and polyethylene glycol.

13. The process according to claim 11, wherein the at least one plasticizing agent is present in the anhydrous composition in an amount ranging from 0.05 to 20% by weight relative to the total weight of the anhydrous composition.

14. The process according to claim 1, wherein the anhydrous composition further comprises at least one direct dye.

15. The process according to claim 14, wherein the at least one direct dye is present in the anhydrous composition in an amount ranging from 0 to 3% by weight relative to the weight of the anhydrous composition.

16. The process according to claim 1, wherein the film has a thickness ranging from 10 to 2000 μm.

17. The process according to claim 16, wherein the film has a thickness ranging from 20 to 500 μm.

18. The process according to claim 1, wherein the anhydrous composition comprises water in an amount less than 10% by weight relative to the total weight of the anhydrous composition.

19. The process according to claim 18, wherein the anhydrous composition comprises water in an amount less than 5% by weight relative to the total weight of the anhydrous composition.

20. The process according to claim 19, wherein the anhydrous composition comprises water in an amount less than 3% by weight relative to the total weight of the anhydrous composition.

21. The process according to claim 1, wherein the anhydrous composition further comprises at least one pH regulating agent.

22. The process according to claim 1, wherein the anhydrous composition is deposited on a water-insoluble support.

23. The process according to claim 22, wherein the water-insoluble support is chosen from polyurethanes; thermoplastic elastomers of the styrene-butadiene-styrene, styrene-ethylene-butadiene-styrene, ethylene-vinyl acetate or coether ester type; polyethylenes; polypropylenes; silicones; metal sheets or films; polyamide copolymers comprising polyether blocks; poly(vinylidene chloride); nylon; elastomers of isobutylene-styrene or styrene-isoprene type; and non-woven materials.

24. The process according to claim 23, wherein the metal sheets or films are chosen from aluminum and composite sheets or films comprising polytetrafluoroethylene.

25. The process according to claim 23, wherein the non-woven materials are chosen from cellulose, viscose, cotton and synthetic fibers.

26. The process according to claim 1, wherein the aqueous medium comprises at least one oxidation dye and/or at least one direct dye.

27. The process according to claim 1, wherein the aqueous medium comprises at least one oxidizing agent.

28. The process according to claim 1, wherein the aqueous medium comprises at least one alkaline agent.

29. The process according to claim 1, wherein said treatment is chosen from coloring the keratinous fibers, bleaching the keratinous fibers, and permanently deforming the keratinous fibers.

30. A process for preparing an anhydrous composition in the form of a film, wherein the anhydrous composition comprises at least one film-forming polymer and at least one oxidizing agent, said process comprising:
applying a precursor composition to a support, wherein the precursor comprises a mixture of the at least one film-forming polymer and the at least one oxidizing agent in an appropriate solvent, and
evaporating the solvent.

31. The process according to claim 30, wherein the solvent is present in the precursor composition in an amount ranging from 10 to 95% by weight relative to the total weight of the precursor composition.
